(19) United States
(12) Patent Application Publication
(10) Pub. No.: US 2015/0126655 A1
(43) Pub. Date: May 7, 2015

(54) CATECHOL BASED DIESTERS FOR GENERAL PURPOSE PLASTICIZERS

(71) Applicant: ExxonMobil Research and Engineering Company, Annandale, NJ (US)

(72) Inventors: JIHAD MOHAMMED DAKKA, Whitehouse Station, NJ (US); WEI TANG, New York, NY (US); CHRISTINE A. COSTELLO, Easton, PA (US); PIERRE J. OSTERRIETH, Bruxelles (BE); DIANA S. SMIRNOVA, High Bridge, NJ (US); STEPHEN ZUSHMA, Clinton, NJ (US); CATHERINE ANNE FALER, Houston, TX (US)

(73) Assignee: ExxonMobil Research and Engineering Company, Annandale, NJ (US)

(21) Appl. No.: 14/069,848

(22) Filed: Nov. 1, 2013

(51) Int. Cl. C08K 5/11; C07C 67/11; C07C 67/08; C07C 69/604

(52) U.S. Cl. CPC C08K 5/11; C07C 69/604; C07C 67/11; C07C 67/08; C08K 2201/014
USPC 524/287; 106/505; 560/146

(57) ABSTRACT

An asymmetric diester of catechol having different alkyl groups in each ester moiety. The asymmetric diester can have a first ester moiety of 8 carbon atoms and a second ester moiety of 10 carbon atoms, and a chemical formula as follows:

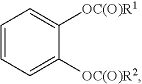

wherein R¹ is C₇H₁₅ and R² is C₉H₁₉, and finds use as a plasticizer compound for polymer compositions.

### FIELD

This disclosure is related to a potential route to non-phthalate, catechol-based diester plasticizers.

### BACKGROUND

Plasticizers are incorporated into a resin (usually a plastic or elastomer) to increase the flexibility, workability, or dispensability of the resin. The largest use of plasticizers is in the production of “plasticized” or flexible polyvinyl chloride (PVC) products. Typical uses of plasticized PVC include films, sheets, tubing, coated fabrics, wire and cable insulation and jacketing, toys, flooring materials such as vinyl sheet flooring or vinyl floor tiles, adhesives, sealants, inks, and medical products such as blood bags and tubing, and the like.

Other polymer systems that use small amounts of plasticizers include polyvinyl butyral, acrylic polymers, nylon, polyolefins, polyurethanes, and certain fluoroplastics. Plasticizers can also be used with rubber (although often these materials fall under the definition of extenders for rubber rather than plasticizers). A listing of the major plasticizers and their compatibilities with different polymer systems is provided in “Plasticizers,” A. D. Godwin, in Applied Polymer Science 21st Century, edited by C. D. Craver and C. E. Carraher, Elsevier (2000); pp. 157-175.

Plasticizers can be characterized on the basis of their chemical structure. The most important chemical class of plasticizers is phthalic acid esters, which accounted for 85% worldwide of PVC plasticizer usage in 2002. However, in the recent past there has been an effort to decrease the use of phthalate esters as plasticizers in PVC, particularly in end uses where the product contacts food, such as bottle cap liners and sealants, medical and food films, or for medical examination gloves, blood bags, and IV delivery systems, flexible tubing, or for toys, and the like. For these and most other uses of plasticized polymer systems, however, a successful substitute for phthalate esters has heretofore not materialized.

One such suggested substitute for phthalates are esters based on cyclohexanoic acid. In the late 1990's and early 2000's, various compositions based on cyclohexanoate, cyclohexanedioates, and cyclohexanepolyoate esters were said to be useful for a range of goods from semi-rigid to highly flexible materials. See, for instance, WO 99/32427, WO 2004/046078, WO 2003/029339, WO 2004/046078, U.S. Application No. 2006-0247461, and U.S. Pat. No. 7,297,738.

Other suggested substitutes include esters based on benzoic acid (see, for instance, U.S. Pat. No. 6,740,254, and also co-pending, commonly-assigned, U.S. Provisional Patent Application No. 61/040,480, filed Mar. 28, 2008 and polyketones, such as described in U.S. Pat. No. 6,777,514; and also co-pending, commonly-assigned, U.S. Patent Publication No. 2008/0242895, filed Mar. 28, 2008. Epoxidized soybean oil, which has much longer alkyl groups (C₁₆ to C₁₈) has been tried as a plasticizer, but is generally used as a PVC stabilizer. Stabilizers are used in much lower concentrations than plasticizers. Co-pending and commonly assigned U.S. Provisional Patent Application No. 61/203,626, filed Dec. 24, 2008, discloses triglycerides with a total carbon number of the triester groups between 20 and 25, produced by esterification of glycerol with a combination of acids derived from the hydroformylation and subsequent oxidation of C₃ to C₉ olefins, having excellent compatibility with a wide variety of resins and that can be made with a high throughput.

JP 62-205140 discloses a plasticizer which is a catechol dicarboxylic ester of the formula:

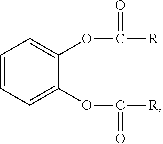

where R is one selected from alkyl, alkenyl, cycloalkyl, aralkyl, aralkenyl, haloalkyl and aryl.

U.S. Pat. Nos. 4,745,026 and 4,833,023 disclose thermal delayed tack sheets prepared by coating a base sheet with a thermal delayed tack composition containing an adhesive polymer, a solid plasticizer, and preferably a tackifier, which plasticizers are fine particle solid compound(s) at room temperature. The solid plasticizer can be a catechol diester.

What is needed is a method of making other general purpose non-phthalate plasticizers having suitable melting or chemical and thermal stability, pour point, glass transition, increased compatibility, good performance and low temperature properties.

### SUMMARY

In one aspect, the present application is directed to an asymmetric diester of catechol having different alkyl groups in each ester moiety, for example wherein a first ester moiety has 8 carbon atoms and a second ester moiety has 10 carbon atoms.

In a preferred embodiment, the asymmetric diester is one having the formula:

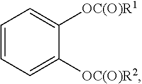

wherein R¹ is C₇H₁₅ and R² is C₉H₁₉, and R¹ and R² can be linear alkyl or branched alkyl.

A further embodiment of the present application is directed to a plasticizer composition comprising an asymmetric diester of catechol having different alkyl groups in each ester moiety, for example wherein said asymmetric diester has a first ester moiety having 8 carbon atoms and a second ester moiety having 10 carbon atoms.

In a preferred embodiment, the asymmetric diester in the plasticizer composition is one having the formula:

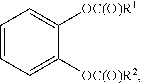

wherein R¹ is C₇H₁₅ and R² is C₉H₁₉, and R¹ and R² can be linear alkyl or branched alkyl.

Conveniently, the plasticizer composition is one further comprising one or more symmetric catechol diesters, each having two identical diester moieties, and can comprise those having two different symmetric catechol diesters, a first of which has ester moieties containing 8 carbon atoms and a second of which has ester moieties containing 10 carbon atoms.

In another embodiment, the present application is directed to a polymer composition comprising a plasticizer composition comprising an asymmetric diester of catechol having different alkyl groups in each ester moiety, and a thermoplastic polymer.

In preferred embodiments, the polymer composition is one wherein the thermoplastic polymer is selected from the group consisting of vinyl chloride resins, polyesters, polyurethanes, ethylene-vinyl acetate copolymer, rubbers, poly(meth)acrylics and combinations thereof, and/or wherein said asymmetric diester has a first ester moiety having 8 carbon atoms and a second ester moiety having 10 carbon atoms.

Advantageously, the asymmetric diester in the polymer composition has the formula:

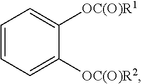

wherein R¹ is C₇H₁₅ and R² is C₉H₁₉, and R¹ and R² can be linear alkyl or branched alkyl.

Conveniently, the polymer composition further comprises one or more symmetric catechol diesters, each having two identical diester moieties, and can have two different symmetric catechol diesters, a first of which has ester moieties containing 8 carbon atoms and a second of which has ester moieties containing 10 carbon atoms.

Another embodiment of the present application is directed to a process for preparing an asymmetric diester of catechol, comprising contacting catechol with a mixture of two different esterifying agents under esterification conditions.

Conveniently, the esterifying agents can be of the formulae:

R¹C(O)Cl and R²C(O)Cl,

wherein R¹ is C₇H₁₅ and R² is C₉H₁₉; or of the formulae:

R¹C(O)OH and R²C(O)OH,

wherein R¹ is C₇H₁₅ and R² is C₉H₁₉, and in either case R¹ and R² can be linear alkyl or branched alkyl.

### BRIEF DESCRIPTION OF THE DRAWINGS

FIG. 1 is a ¹H NMR of the product of Sample 1.

FIG. 2 is a ¹H NMR of the product of Sample 3(b).

FIG. 3 is a gas chromatogram (GC) of the product of Sample 3(b).

FIG. 4 is a graph illustrating the volatility differences between various conventional plasticizers and those according to the present application.

### DETAILED DESCRIPTION

All numerical values within the detailed description and the claims herein are modified by “about” or “approximately” the indicated value, and take into account experimental error and variations that would be expected by a person having ordinary skill in the art.

There is an increased interest in developing new plasticizers that are non-phthalates and which possess good plasticizer performance characteristics but are still competitive economically. The present disclosure is directed towards non-phthalate ester plasticizers, particularly OXO-ester plasticizers, that can be made from low cost feeds and employ fewer manufacturing steps in order to meet economic targets.

The present application is directed to an asymmetric diester of catechol having different alkyl groups in each ester moiety. In the present application, the term “asymmetric diester” means that the two ester moieties on the molecule are different, for example wherein a first ester moiety has 8 carbon atoms and a second ester moiety has 10 carbon atoms.

It has been determined that compounds of the general formula:

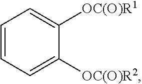

wherein R¹ is C₇H₁₅ and R² is C₉H₁₉, are particularly useful as replacements for diisononylphthalate (DINP) as plasticizers for conventional polymer plastics. In a preferred embodiment, R¹ and R² can be linear alkyl, i.e. the hydrocarbon residue of a n-carboxylic acid or n-acyl halide, or branched alkyl, i.e. the hydrocarbon residue of a branched carboxylic acid or branched acyl halide, such as those formed by or from OXO-acids, discussed in more detail below.

One route to non-phthalate plasticizers of the present disclosure is by contacting catechol, which is commercially available, with a mixture of two different esterifying agents under esterification conditions. The esterifying agents can be of the formulae:

R¹C(O)Cl and R²C(O)Cl,

wherein R¹ is C₇H₁₅ and R² is C₉H₁₉, wherein R¹ and R² can be linear alkyl or branched alkyl.; or of the formulae:

R¹C(O)OH and R²C(O)OH,

wherein R¹ is C₇H₁₅ and R² is C₉H₁₉, wherein R¹ and R² can be linear alkyl or branched alkyl.

In order to obtain the asymmetric diesters of the present disclosure, it is advantageous to contact the catechol with a mixture of esterifying agents having different alkyl-chain lengths. In this manner, some of the hydroxyl moieties are esterified with one of the esterifying agents, and some of the hydroxyl moieties are esterified with the other esterifying agent. Of course, according to this reaction scheme, a certain amount of the catechol reactant will be di-esterified with identical esterifying agents, such that significant amounts of symmetric catechol diesters are also produced.

Accordingly, when the esterifying agent is an acyl halide, such as an acyl chloride, the esterification reaction proceeds as follows:

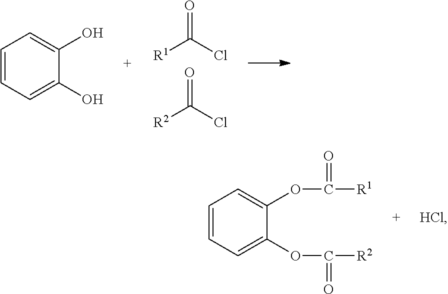

wherein R¹ and R² are as described above.

Alternatively, when the esterifying agent is a carboxylic acid, the esterification reaction proceeds as follows:

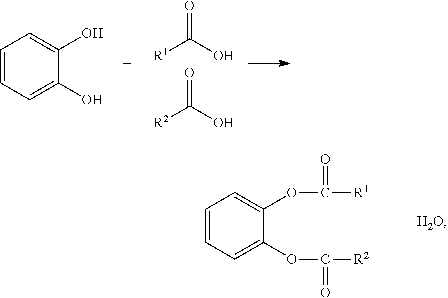

wherein R¹ and R² are as described above.

In either reaction scheme, the resulting asymmetric diester of catechol is formed, usually in combination with symmetric diesters of catechol of the formulae:

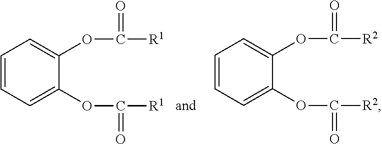

in varying ratios.

For example, a plasticizer composition is produced containing an asymmetric diester of the general formula:

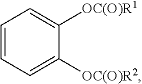

wherein R¹ is C₇H₁₅ and R² is C₉H₁₉, as described above, and one or more symmetric catechol diesters, each having two identical diester moieties, such as those having two different symmetric catechol diesters, a first of which has ester moieties containing 8 carbon atoms and a second of which has ester moieties containing 10 carbon atoms.

According to the present application an “OXO-acid” is an organic acid, or mixture of organic acids, which is prepared by hydroformylating an olefin, followed by oxidation to form the acids. Typically, the olefin is formed by light olefin oligomerization over heterogenous acid catalysts, which olefins are readily available from refinery processing operations. The reaction results in mixtures of longer-chain, branched olefins, which subsequently form longer chain, branched alcohols or acids, as described in U.S. Pat. No. 6,274,756, incorporated herein by reference in its entirety. The OXO-alcohols consist of multiple isomers of a given chain length due to the various isomeric olefins obtained in the oligomerization process, in tandem with the multiple isomeric possibilities of the hydroformylation step. The OXO-acids similarly consist of multiple isomers of a given chain length.

According to the present specification an “OXO-ester” is a compound having at least one functional ester moiety within its structure derived from esterification of either an acid or alcohol compound with an OXO-alcohol or OXO-acid, respectively. In the present application, an OXO-ester can be formed by esterification of catechol, a dialcohol, with one or more OXO-acids or acyl halide derivatives thereof.

“Hydroformylating” or “hydroformylation” is the process of reacting a compound having at least one carbon-carbon double bond (an olefin) in an atmosphere of carbon monoxide and hydrogen over a cobalt or rhodium catalyst, which results in addition of at least one aldehyde moiety to the underlying compound. U.S. Pat. No. 6,482,972, which is incorporated herein by reference in its entirety, describes the hydroformylation (OXO) process.

Branched aldehydes can be produced by hydroformylation of C₃ to C₁₂ olefins; in turn, some of these olefins have been produced by propylene and/or butene oligomerization over solid phosphoric acid or zeolite catalysts. The resulting C₄ to C₁₄ aldehydes can then be recovered from the crude hydroformylation product stream by fractionation to remove unreacted olefins. These C₄ to C₁₃ aldehydes can then hydrogenated to alcohols (OXO-alcohols) or oxidized to acids (OXO-acids). Single carbon number acids or alcohols can be used in the esterification of the aromatic acids described above, or differing carbon numbers can be used to optimize product cost and performance requirements. The “OXO” technology provides cost advantaged alcohols and acids. Other options are considered, such as hydroformylation of C₄-olefins to C₅-aldehydes, followed by hydrogenation to C₅-alcohols, or aldehyde dimerization followed by hydrogenation or oxidation to C₁₀ alcohols or acids. It is understood that the term “branched” describes the overall isomeric mixture of the aldehydes (and subsequent acids, alcohols, and residues thereof). Thus, a “branched” OXO-aldehyde, acid, alcohol, or residue contains some portion of linear isomers mixed in with the individual branched isomers.

“Hydrogenating” or “hydrogenation” is addition of hydrogen (H₂) to a double-bonded functional site of a molecule, such as in the present case the addition of hydrogen to the aldehyde to form the corresponding alcohol. Conditions for hydrogenation of an aldehyde are well-known in the art and include, but are not limited to temperatures of 0-300° C., pressures of 1-500 atmospheres, and the presence of homogeneous or heterogeneous hydrogenation catalysts such as Pt/C, Pt/Al₂O₃ or Pd/Al₂O₃.

Alternatively, the OXO-acids or OXO-alcohols can be prepared by aldol condensation of shorter-chain aldehydes to form longer chain aldehydes, as described in U.S. Pat. No. 6,274,756, followed by oxidation or hydrogenation to form the OXO-acids or OXO-alcohols, respectively.

“Esterifying” or “esterification” is reaction of a carboxylic acid moiety, including acyl halides or anhydrides, with an organic alcohol moiety to form an ester linkage. Esterification conditions are well-known in the art and include, but are not limited to, temperatures of 0-300° C., and the presence or absence of homogeneous or heterogeneous esterification catalysts such as Lewis or Brønsted acid catalysts.

As discussed above, the resulting OXO-acids can be used individually or together in mixtures having different chain lengths, or in isomeric mixtures of the same carbon chain length to make mixed esters for use as plasticizers. This mixing of carbon numbers and/or levels of branching can be advantageous to achieve the desired compatibility with PVC for the respective core acid used for the polar moiety end of the plasticizer, and to meet other plasticizer performance properties.

The overall isomeric distribution of the OXO-acids may be described quantitatively by parameters such as average branch content per molecule or per chain position. Branching may be determined by Nuclear Magnetic Resonance (NMR) spectroscopy.

Typical branching characteristics of OXO-acids are provided in Table 1, below.

TABLE 1
¹³C NMR Branching Characteristics of Typical OXO-Acids.

| OXO- | Average | Pendant | Total | Pendant | % Carbonyls α to |
|---|---|---|---|---|---|
| Acid | Carbon No. | Methyls^{a} | Methyls^{b} | Ethyls | Branch^{c} |
| C₄^{d} | 4.0 | 0.35 | 1.35 | 0 | 35 |
| C₅^{e} | 5.0 | 0.35 | 1.35 | 0 | 30 |
| C₆ | — | — | — | — | — |
| C₇ | 6.88-7.92 | 0.98-1.27 | 1.94-2.48 | 0.16-0.26 | 11.3-16.4 |
| C₈ | 8.1-8.3 | — | 2.7 | — | 12-15 |
| C₉ | 9.4 | — | n/a | — | 12 |
| C₁₀ | 10.2 | — | n/a | — | 12 |
| C₁₂ | — | — | | — | — |
| C₁₃ | 12.5 | — | 4.4 | — | 11 |

— Data not available.
^{a}C₁ Branches only.
^{b}Includes methyls on all branch lengths and chain end methyls.
^{c}The “alpha” position in the acid nomenclature used here is equivalent to the alcohol “beta” carbon in Table 1.
^{d}Calculated values based on an assumed molar isomeric distribution of 65% n-butanoic acid and 35% isobutanoic acid (2-methylpentanoic acid).
^{e}Calculated values based on an assumed molar isomeric distribution of 65% n-pentanoic acid, 30% 2-methylbutanoic acid, and 5% 3-methylbutanoic acid.

In general, for every polymer to be plasticized, a plasticizer is required with the correct balance of solubility, volatility and viscosity to have acceptable plasticizer compatibility with the resin. In particular, if the 20° C. kinematic viscosity is higher than 250 mm²/sec as measured by the appropriate ASTM test, or alternately if the 20° C. cone-and-plate viscosity is higher than 250 cP, this will affect the plasticizer processability during formulation, and can require heating the plasticizer to ensure good transfer during storage and mixing of the polymer and the plasticizer. Volatility is also a very critical factor which affects the long-term plasticizer formulation stability. Higher volatility plasticizers can migrate from the plastic resin matrix and cause damage to the article. The plasticizer volatility in a resin matrix can be roughly predicted by neat plasticizer weight loss at 220° C. using Thermogravimetric Analysis.

We have found that when C₈ and C₁₀ OXO-acids or acyl halides are used as reactants for the esterification reactions described above, the resulting OXO-esters are in the form of relatively high-boiling liquids (having low volatility), which are readily incorporated into polymer formulations as plasticizers.

Accordingly, another embodiment of this disclosure is directed to a polymer composition comprising a thermoplastic polymer and at least one plasticizer of the formula:

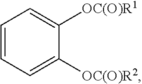

wherein R¹ is C₇H₁₅ and R² is C₉H₁₉, and R¹ and R² can be linear alkyl or branched alkyl, and in which the thermoplastic polymer can be selected from the group consisting of vinyl chloride resins, polyesters, polyurethanes, ethylene-vinyl acetate copolymer, rubbers, poly(meth)acrylics and combinations thereof.

The following examples are meant to illustrate the present disclosure and inventive processes, and provide where appropriate, a comparison with other methods, including the products produced thereby. Numerous modifications and variations are possible and it is to be understood that within the scope of the appended claims, the disclosure can be practiced otherwise than as specifically described herein.

Accordingly, in a first embodiment the disclosure is directed to an asymmetric diester of catechol having different alkyl groups in each ester moiety.

In a second embodiment, the asymmetric diester of the first embodiment comprises a first ester moiety which has 8 carbon atoms and a second ester moiety which has 10 carbon atoms.

In a third embodiment, the asymmetric diester of either the first embodiment or the second embodiment, can have the formula:

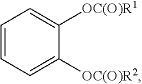

wherein R¹ is C₇H₁₅ and R² is C₉H₁₉.

Advantageously, in a fourth embodiment the asymmetric diester of the third embodiment, can be formulated such that R¹ and R² are linear alkyl or branched alkyl.

In a fifth embodiment, the present disclosure is directed to a plasticizer composition comprising an asymmetric diester of catechol having different alkyl groups in each ester moiety.

Preferably, according to a sixth embodiment, the plasticizer composition of the fifth embodiment comprises said asymmetric diester which has a first ester moiety having 8 carbon atoms and a second ester moiety having 10 carbon atoms.

In a seventh embodiment, according to either the fifth or sixth embodiment said asymmetric diester has the formula:

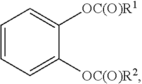

wherein R¹ is C₇H₁₅ and R² is C₉H₁₉.

In an eighth embodiment, the diester of the seventh embodiment can be formulated such that R¹ and R² of said asymmetric diester are linear alkyl or branched alkyl.

In a ninth embodiment, the composition of any of the fifth through eighth embodiments can further comprise one or more symmetric catechol diesters, each having two identical diester moieties.

In a tenth embodiment, the composition of embodiment 9 comprises two different symmetric catechol diesters, a first of which has ester moieties containing 8 carbon atoms and a second of which has ester moieties containing 10 carbon atoms.

In an eleventh embodiment, the present disclosure is directed to a polymer composition comprising a plasticizer composition comprising an asymmetric diester of catechol having different alkyl groups in each ester moiety, and a thermoplastic polymer.

In a twelfth embodiment, the thermoplastic polymer of the eleventh embodiment can be selected from the group consisting of vinyl chloride resins, polyesters, polyurethanes, ethylene-vinyl acetate copolymer, rubbers, poly(meth)acrylics and combinations thereof.

In a thirteenth embodiment, the polymer composition according to either of the eleventh or twelfth embodiments can comprise said asymmetric diester which has a first ester moiety having 8 carbon atoms and a second ester moiety having 10 carbon atoms.

In a fourteenth embodiment, the polymer composition of any of the eleventh through thirteenth embodiments can have said asymmetric diester having the formula:

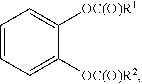

wherein R¹ is C₇H₁₅ and R² is C₉H₁₉, and R¹ and R² are linear alkyl or branched alkyl.

In a fifteenth embodiment, the polymer composition of any of the eleventh through fourteenth embodiments can further comprise one or more symmetric catechol diesters, each having two identical diester moieties.

In a sixteenth embodiment, the polymer composition of embodiment 15 comprises two different symmetric catechol diesters, a first of which has ester moieties containing 8 carbon atoms and a second of which has ester moieties containing 10 carbon atoms.

In a seventeenth embodiment, the present disclosure is directed to a process for preparing an asymmetric diester of catechol, comprising contacting catechol with a mixture of two different esterifying agents under esterification conditions.

According to an eighteenth embodiment, the process of embodiment 17 utilizes esterifying agents are of the formulae:

R¹C(O)Cl and R²C(O)Cl,

wherein R¹ is C₇H₁₅ and R² is C₉H₁₉, and R¹ and R² are linear alkyl or branched alkyl.

Alternatively, in a nineteenth embodiment, the esterifying agents useful in embodiment 17 are of the formulae:

R¹C(O)OH and R²C(O)OH,

wherein R¹ is C₇H₁₅ and R² is C₉H₁₉, and R¹ and R² are linear alkyl or branched alkyl.

### EXAMPLES

### Sample 1

### Synthesis of Catechol Based Plasticizer from Catechol with Octanoyl Chloride

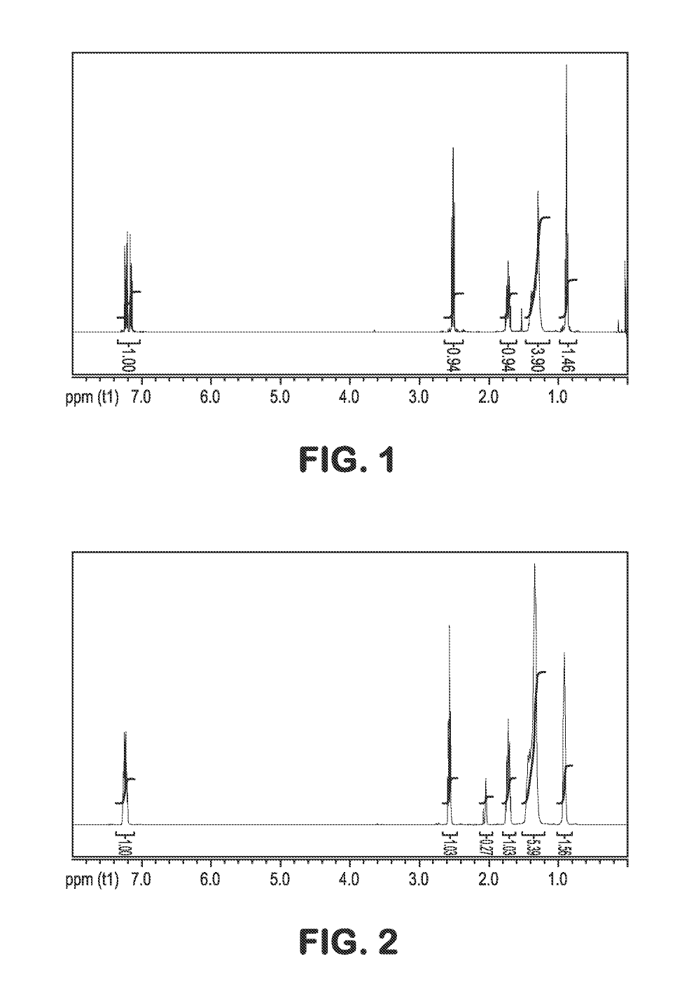

Catechol (25 g, 0.227 mol) was dissolved into 250 ml acetone in a 1 L round bottom flask. Triethylamine (65 ml) was added to the flask and the solution was purged with nitrogen for 40 min. Octanoyl chloride (94.6 ml, 0.454 mol) was added dropwise over 1 hour. The solution was then heated to 50° C. for 5 hours. The reaction was stopped and the product filtered, washed with sodium hydroxide 2 times and with a saturated salt solution 2 times. The solvent was then removed via rotavap and the final product was distilled under vacuum. The ¹H NMR of the product is shown in FIG. 1, which is a catechol C₈C₈ diester.

### Sample 2

### Synthesis of Catechol Based Plasticizer from Catechol with Decanoyl Chloride

The procedure of Sample 1 was followed except that octanoyl chloride was replaced with decanoyl chloride (38.7 ml, 0.227 mol), to form a catechol C₁₀C₁₀ diester.

### Samples 3(a)-(c)

### Synthesis of Catechol Based Plasticizer from Catechol with Mixed Octanol Chloride and Decanoyl Chloride

The procedure of Sample 1 was followed except that octanoyl chloride was replaced with mixtures of octanoyl chloride (47.3 ml, 0.227 mol) and decanoyl chloride (38.7 ml, 0.227 mol). After purification, the products had the following catechol diester compositions.

3(a): C₈C₈ (21.7%); C₈C₁₀ (48.9%); C₁₀C₁₀ (29.4%)

3(b): C₈C₈ (14.0%); C₈C₁₀ (48.8%); C₁₀C₁₀ (37.2%)

3(c): C₈C₈ (20.4%); C₈C₁₀ (52.0%); C₁₀C₁₀ (27.6%)

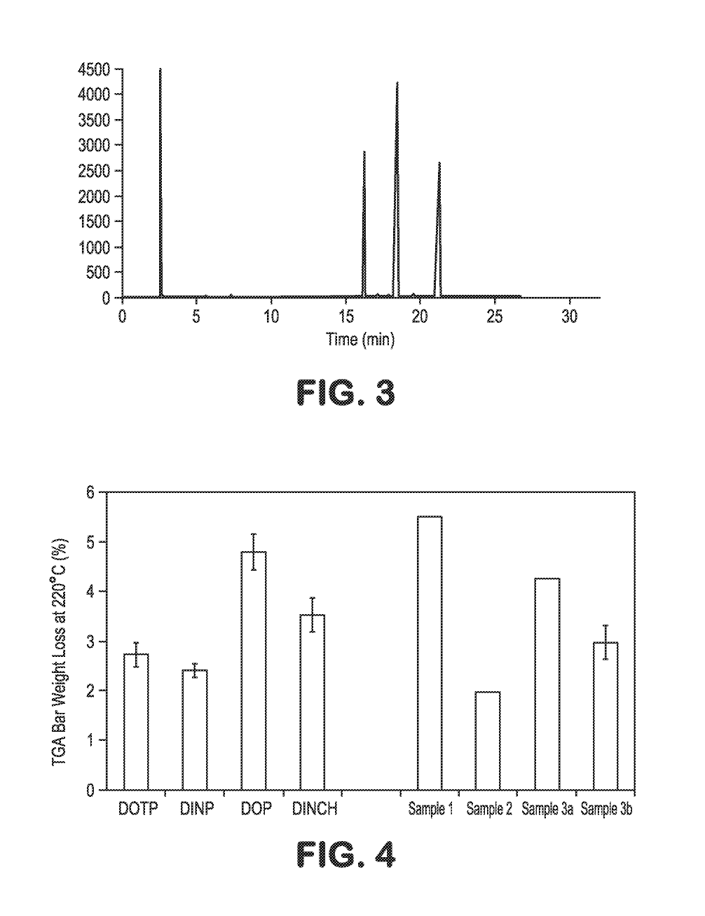

The ¹H NMR of Sample 3(b) is shown in FIG. 2 and its gas chromatogram is shown in FIG. 3.

### Testing Data

The catechol diesters of Samples 1-3 above were subjected to testing for comparison of their properties against a number of conventional plasticizers. Plasticizers were tested in both neat and compound form.

### Neat Ester Evaluation by Differential Scanning Calorimetry, Thermogravimetric Analysis and Viscosity

Thermogravimetric Analysis (TGA) was conducted on the neat esters using a TA Instruments TGA Q5000 instrument (25-450° C., 10° C./min, under 25 cc N₂/min flow through furnace and 10 cc N₂/min flow through balance; sample size approximately 10 mg). Table 2, below, provides a volatility comparison of the different esters. Differential Scanning Calorimetry (DSC) was also performed on the neat esters, using a TA Instruments Q2000 calorimeter fitted with a liquid N₂ cooling accessory. Samples were loaded at room temperature and cooled to −110° C. at 10° C./min and analyzed on heating to 75° C. at a rate of 10° C./min. Table 2 provides a glass transition (T_{g}) comparison of the different esters. T_{g}s given in Table 2 are midpoints of the first heats (unless only one heat cycle was performed, in which case the first heat T_{g}, which is typically in very close agreement, is given). Kinematic Viscosity (KV) was measured at 20° C. according to ASTM D-445-20; results are summarized in Table 4. Comparative data for a common commercial plasticizer (diisononyl phthalate; Jayflex® DINP, ExxonMobil Chemical Co.) is also included.

TABLE 2
Volatility, Viscosity, and Glass Transition Properties of Neat Esters

| | TGA | TGA | TGA | TGA | | KV |
|---|---|---|---|---|---|---|
| | 1% | 5% | 10% | Wt Loss | DSC | (20° C., |
| | Wt Loss | Wt Loss | Wt Loss | at 220° C. | T_{g} | mm²/ |
| Ex. No. | (° C.) | (° C.) | (° C.) | (%) | (° C.) | sec) |
| DINP | 184.6 | 215.2 | 228.5 | 6.4 | −79.1 | 96.81 |
| Samp. 1 | 153.8 | 188.1 | 204.6 | 18.3 | −103.8 | |
| Samp. 2 | 138.9 | 217.0 | 235.1 | 5.5 | 50.8 | |
| Samp. 3(a) | 73.7 | 180.7 | 205.2 | 15.3 | −88.5 | |
| Samp. 3(b) | — | — | — | — | — | |
| Samp. 3(c) | 171.9 | 204.7 | 220.9 | 9.6 | −84.0 | |

— Data not obtained.

### General Procedure Melt Mixing Ester and PVC

In a 250 ml beaker is added 2.7 g of an additive package containing a 70/30 wt/wt of Paraplex G62 ESO/Mark 4716. To this is added 19.1 g of plasticizer and the mixture is stirred with a spatula until blended. After blending, 38.2 g of PVC is added and the mixture is mixed forming a paste. The mixture is added to the melt mixture. A Haake Rheomix 600 mixer manufactured by Haake PolyLab System is preheated to the desired mixing temperature (165° C. for most experiments). A coarsely mixed sample consisting of plasticizer, polyvinylchloride and stabilizers is added to the mixer while stirring at 35 rpm. After addition the mixer is stopped for one minute. The mixer is started again and the sample is mixed for five minutes. After mixing for five minutes the mixer is stopped and disassembled. The mixed sample is removed hot.

98° C. Weight Loss Comparison of PVC Bars Plasticized with Esters Versus PVC Bars Plasticized with Commercial Plasticizer

Two each of the PVC sample bars prepared above were placed separately in aluminum weighing pans and placed inside a convection oven at 98° C. Initial weight measurements of the hot bars and measurements taken at specified time intervals were recorded and results were averaged between the bars. The averaged results are shown in Table 3. Notes on the appearance and flexibility of the bars at the end of the test are also given.

TABLE 3
98° C. % Weight Loss of Ester-Containing PVC Bars and
DINP-Containing PVC Control Bars.

| Example No. | | | | | |
|---|---|---|---|---|---|
| (Ester Used in Bar) | Day 1 | Dav 7 | Day 14 | Day 21 | Notes on Bar |
| DINP | 0.31 | 0.48 | 0.64 | 0.74 | Light brown; |
| | | | | | good flex |
| Samp. 1 | 0.43 | 1.4 | 2.3 | 3.0 | |
| Samp. 2 | 0.35 | 0.65 | 0.82 | 1.0 | |
| Samp. 3(a) | 0.74 | 1.8 | 2.5 | 2.7 | |
| Samp. 3(b) | 0.30 | 1.0 | 1.3 | 1.6 | |
| Samp. 3(c) | — | — | — | — | |

— Data not obtained.

Demonstration of Plasticization of PVC with Esters Via Thermogravimetric Analysis (TGA) and Differential Scanning Calorimetry (DSC).

Thermogravimetric Analysis (TGA) was conducted on the neat esters using a TA Instruments TGA5000 instrument (25-450° C., 10° C./min, under 25 cc N₂/min flow through furnace and 10 cc N₂/min flow through balance; sample size approximately 10 mg). Table 4 provides a volatility comparison of the neat and plasticized PVC bars. Differential Scanning Calorimetry (DSC) was performed on the compression-molded sample bars (PVC:plasticizer ratio 2:1) using a TA Instruments Q2000 calorimeter fitted with a liquid N₂ cooling accessory. Samples were loaded at room temperature and cooled to approximately −90° C. at 10° C./min, and then analyzed on heating at a rate of 10° C./min to 100-150° C. for plasticized PVC bars, and to 100° C. for the comparative neat PVC bar. Small portions of the sample bars (typical sample mass 5-7 mg) were cut for analysis, making only vertical cuts perpendicular to the largest surface of the bar to preserve the upper and lower compression molding “skins”; the pieces were then placed in the DSC pans so that the upper and lower “skin” surfaces contacted the bottom and top of the pan. Table 5 provides the first heat T_{g} onset, midpoint, and end for neat PVC and the plasticized bars. A lowering and broadening of the glass transition for neat PVC is observed upon addition of the esters, indicating plasticization and extension of the flexible temperature range of use for neat PVC. The data in Table 5 provides a measure of the flexibility range of the plasticized PVC specimen, measured by DSC. The range of the glass transition corresponds to the flexibility range. Most advantageous are samples which demonstrate both a broad T_{g} range (the difference between T_{g}, onset and T_{g}, end) as well as having a low flex onset (as measured by T_{g}, onset).

TABLE 4
Volatility Properties of Neat PVC and PVC Sample Bars Plasticized Esters.

| | TGA 1% Wt | TGA 5% Wt | TGA 10% Wt | TGA % Wt Loss at |
|---|---|---|---|---|
| Ex. No. | Loss (° C.) | Loss (° C.) | Loss (° C.) | 220° C. |
| NONE | 129.9 | 192.3 | 255.4 | 6.3 |
| (neat PVC) | | | | |
| DINP | 204.6 | 247.4 | 257.6 | 1.8 |
| Samp. 1 | 175.3 | 217.7 | 240.3 | 5.5 |
| Samp. 2 | 196.6 | 240.6 | 249.0 | 2.0 |
| Samp. 3(a) | 174.4 | 225.6 | 242.8 | 4.3 |
| Samp. 3(b) | 190.7 | 234.2 | 244.5 | 3.0 |
| Samp. 3(c) | — | — | — | — |

— Data not obtained.

FIG. 4 shows that volatility of catechol diesters with mixed alkyl chains, Sample 3(b), is only slightly worse than DINP.

TABLE 5
Glass Transition Onset, Midpoint,
and End for Plasticized PVC Bars by DSC.

| | T_{g} Onset | T_{g} Midpt | T_{g} End | Tₘ, pk |
|---|---|---|---|---|
| Ex. No. | (° C.) | (° C.) | (° C.) | (° C.) |
| NONE | 44.5 | 46.4 | 48.9 | not calc. |
| (neat PVC) | | | | |
| DINP | −37.8 | −24.8 | −12.2 | not calc. |
| Samp. 1 | −41.5 | −22.6 | −3.6 | 57.1 |
| Samp. 2 | −48.0 | −26.7 | −5.1 | 61.1 |
| Samp. 3(a) | −46.9 | −23.3 | 0.2 | 60.4 |
| Samp. 3(b) | −47.3 | −25.3 | −3.3 | 56.5 |
| Samp. 3(c) | — | — | — | — |

— Data not obtained.

The data show that effective non-phthalate plasticizers can be made from catechol which is esterified with a combination of C₈ and C₁₀ esterifying agents.

The meanings of terms used herein shall take their ordinary meaning in the art; reference shall be taken, in particular, to Handbook of Petroleum Refining Processes, Third Edition, Robert A. Meyers, Editor, McGraw-Hill (2004). In addition, all patents and patent applications, test procedures (such as ASTM methods), and other documents cited herein are fully incorporated by reference to the extent such disclosure is not inconsistent with this disclosure and for all jurisdictions in which such incorporation is permitted. Also, when numerical lower limits and numerical upper limits are listed herein, ranges from any lower limit to any upper limit are contemplated. Note further that Trade Names used herein are indicated by a™ symbol or ® symbol, indicating that the names may be protected by certain trademark rights, e.g., they may be registered trademarks in various jurisdictions.

The disclosure has been described above with reference to numerous embodiments and specific examples. Many variations will suggest themselves to those skilled in this art in light of the above detailed description. All such obvious variations are within the full intended scope of the appended claims.

What is claimed is:

1. An asymmetric diester of catechol having different alkyl groups in each ester moiety.

2. The asymmetric diester of claim 1, wherein a first ester moiety has 8 carbon atoms and a second ester moiety has 10 carbon atoms.

3. The asymmetric diester of claim 1, having the formula: 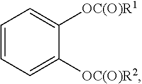
wherein R¹ is C₇H₁₅ and R² is C₉H₁₉.

4. The asymmetric diester of claim 3, wherein R¹ and R² are linear alkyl or branched alkyl.

5. A plasticizer composition comprising an asymmetric diester of catechol having different alkyl groups in each ester moiety.

6. The plasticizer composition of claim 5, wherein said asymmetric diester has a first ester moiety having 8 carbon atoms and a second ester moiety having 10 carbon atoms.

7. The plasticizer composition of claim 5, wherein said asymmetric diester has the formula: 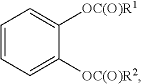
wherein R¹ is C₇H₁₅ and R² is C₉H₁₉.

8. The plasticizer composition of claim 7, wherein R¹ and R² of said asymmetric diester are linear alkyl or branched alkyl.

9. The plasticizer composition of claim 5, further comprising one or more symmetric catechol diesters, each having two identical diester moieties.

10. The plasticizer composition of claim 9, comprising two different symmetric catechol diesters, a first of which has ester moieties containing 8 carbon atoms and a second of which has ester moieties containing 10 carbon atoms.

11. A polymer composition comprising a plasticizer composition comprising an asymmetric diester of catechol having different alkyl groups in each ester moiety, and a thermoplastic polymer.

12. The polymer composition of claim 11, wherein the thermoplastic polymer is selected from the group consisting of vinyl chloride resins, polyesters, polyurethanes, ethylene-vinyl acetate copolymer, rubbers, poly(meth)acrylics and combinations thereof.

13. The polymer composition of claim 11, wherein said asymmetric diester has a first ester moiety having 8 carbon atoms and a second ester moiety having 10 carbon atoms.

14. The polymer composition of claim 11, wherein said asymmetric diester has the formula: 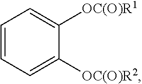
wherein R¹ is C₇H₁₅ and R² is C₉H₁₉, and R¹ and R² are linear alkyl or branched alkyl.

15. The polymer composition of claim 11, further comprising one or more symmetric catechol diesters, each having two identical diester moieties.

16. The polymer composition of claim 15, comprising two different symmetric catechol diesters, a first of which has ester moieties containing 8 carbon atoms and a second of which has ester moieties containing 10 carbon atoms.

17. A process for preparing an asymmetric diester of catechol, comprising contacting catechol with a mixture of two different esterifying agents under esterification conditions.

18. The process of claim 17, wherein the esterifying agents are of the formulae:
R¹C(O)Cl and R²C(O)Cl,
wherein R¹ is C₇H₁₅ and R² is C₉H₁₉, and R¹ and R² are linear alkyl or branched alkyl.

19. The process of claim 17, wherein the esterifying agents are of the formulae:
R¹C(O)OH and R²C(O)OH,
wherein R¹ is C₇H₁₅ and R² is C₉H₁₉, and R¹ and R² are linear alkyl or branched alkyl.
